(19) United States
(12) Patent Application Publication
(10) Pub. No.: US 2003/0212034 A1
(43) Pub. Date: Nov. 13, 2003

(54) Method of treatment of obesity and paralyzed muscle and ergogenic aids

(76) Inventor: William W Winder, Provo, UT (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/297,117

(22) PCT Filed: Jun. 11, 2001
(86) PCT No.: PCT/US01/18812

(51) Int. Cl. A61K 31/7076

(52) U.S. Cl. 514/47

(57) ABSTRACT

The invention relates to a method of treating obesity in a mammal. The method includes the step of administering a therapeutically effective amount of an AMP-activated protein kinase activator to the mammal. The mammal may be for example, a human, a rat, a mouse, and the like. The AMP-activated protein kinase activator can be subcutaneously injected into the mammal or administered in any other manner that provides for uptake of the AMP-activated protein kinase activator into the cells of the mammal. The activation of the AMP-activated protein kinase activator can produce the benefits of exercise training including the loss of body fat. The invention also relates to a method of treating insulin resistance in a mammal suffering from obesity, type 2 diabetes, or muscle paralysis. To reduce the insulin resistance a therapeutically effective amount of an AMP-activated protein kinase activator is given to the mammal.

### 1. RELATED APPLICATIONS

This application is related to and claims the benefit of U.S. Provisional Application Serial No. 60/210,708 of William W. Winder Jun. 9, 2000 and entitled “Use of AMP Kinase Activators for Treatment of Obesity and Paralyzed Muscle and as Ergogenic Aids,” which is incorporated herein by this reference.

### 2. FIELD OF THE INVENTION

The present invention relates to the methods of treatment of obesity and paralyzed muscle. More specifically, the invention relates methods of treatment of obesity and paralyzed muscle through artificial activation of metabolic pathways.

### 3. TECHNICAL BACKGROUND

Obesity is one of the largest health problems in the United States and is a growing concern for many health care officials. By one account more than 33 percent of adults and 20 percent of children in the United States are considered obese. Obesity is defined as having excessive amounts of body fat. Body fat (adipose tissue) is necessary for certain bodily functions. However, when body fat accumulates to excessive amounts the person is considered obese. Obesity can lead to a number of different illness including: heart disease, high blood pressure, increased cholesterol, diabetes, certain types of cancer, orthopedic problems, musculo-skeletal diseases, decreased flexibility, and difficulty breathing.

Obesity can have both genetic and habitual causes. A diet high in fat combined with a sedentary lifestyle can contribute the accumulation of body fat. The most commonly prescribed remedy for obesity is reduced caloric intake and exercise. However, as one becomes increasing obese, the desire and ability to exercise can become significantly reduced. Thus, it can be difficult for an obese person to lose the body fat required to be healthier. Moreover, obesity has been implicated the development of resistance to insulin which causes significant adverse effects to one's health.

Biochemical adaptations of skeletal muscle to endurance exercise have been extensively studied beginning with a report in 1967 showing an increase in mitochondrial oxidative enzyme activities in response to three months of endurance training in rats. Booth, F. W. & K. M. Baldwin In: Handbook of Physiology, Section 12. Exercise: Regulation and Integration of Multiple Systems, edited by L. B. Rowell and J. T. Shepherd, 1996, p. 1075-1123; Holloszy, J. O. J. Biol. Chem. 242:2278-2282, 1967; Holloszy, J. O., & F. W. Booth. Ann. Rev. Physiol. 38:273-291, 1976; Holloszy, J. O. & E. F. Coyle. J. Appl. Physiol. 56:831-838, 1984.

One of the key adaptations to endurance exercise training is the increase in mitochondrial oxidative enzymes of the muscles involved in the exercise. It has been shown that skeletal muscle enzymes of the citric acid cycle, electron transport chain, and also enzymes of fatty acid oxidation all increase in response to an endurance training program or to chronic electrical stimulation. Booth, F. W. & K. M. Baldwin In: Handbook of Physiology, Section 12. Exercise: Regulation and Integration of Multiple Systems, edited by L. B. Rowell and J. T. Shepherd, 1996, p. 1075-1123; Essig, D. A. Exercise Sports Sci. Rev. 24:289-319, 1996; Holloszy, J. O. J. Biol. Chem. 242:2278-2282, 1967; Holloszy, J. O., & F. W. Booth. Ann. Rev. Physiol. 38:273-291, 1976; Holloszy, J. O. & E. F. Coyle. J. Appl. Physiol. 56:831-838, 1984. The physiological consequence of this adaptation is an increase in capacity to oxidize pyruvate and fatty acids and to generate ATP. The insulin-sensitive glucose transporter (GLUT4) and hexokinase tend to adapt in the same direction as the mitochondrial oxidative enzymes. Etgen, G. J., et al. Am. J. Physiol. 272:E864-E869, 1997; Gulve, E. A, & R. J. Spina J. Appl. Physiol. 79:1562-1566, 1995; Hayashi, T., et al. Am. J. Physiol. 273:E1039-E1051, 1997; Holloszy, J. O., & P. A. Hansen Rev. Physiol. Biochem. and Pharm. 128:99-193, 1996; Host, H. H., et al. J. Appl. Physiol. 85:133-138, 1998; Ivy, J. L. Sports Med. 24:321-336, 1997; Neufer, P. D., & G. L. Dohm Am. J. Physiol. 265:C1597-C1603, 1993; Ren, J. M., et al. J. Biol. Chem. 269:14396-14401, 1994; Ren, J. M., et al. Am. J. Physiol. 264:C146-C150, 1993.

Although these adaptations are well-characterized, little is known regarding the mechanisms coupling muscle contractile activity to the increase in expression of these proteins in the muscle. See Essig, D. A. Exercise Sports Sci. Rev. 24:289-319, 1996. A recent study provides evidence that 5′-AMP activated protein kinase may be involved in some of these adaptations. It has also been shown that incubation of epitrochlearis muscle with AICAR for 18 hours results in increases in GLUT4 and hexokinase, providing additional evidence of AMPK involvement in control of muscle gene expression. Ojuka, E. O., et al. J. Appl. Physiol. (In Press), 2000. AMPK activity has been shown to increase in skeletal muscle of rats running on the treadmill and in electrically stimulated muscle. Ruderman, N. B., et al. Am. J. Physiol. 276:E1-E18, 1999; Winder, W. W., & D. G. Hardie Am. J. Physiol. 270:E299-E304, 1996; Winder, W. W., & D. G. Hardie, Am. J. Physiol. 277:E1-10, 1999. These observations taken together suggest that AMPK activation may be involved in mediating the effect of exercise on at least some of the biochemical adaptations of muscle.

AMPK has recently been implicated as being important as a metabolic master switch in the muscle, controlling both fat metabolism and glucose uptake. Winder, W. W., & D. G. Hardie, Am. J. Physiol. 277:E1-10, 1999. This enzyme is controlled by both allosteric and covalent mechanisms. It is activated allosterically by an increase in 5′-AMP and inhibited by ATP and creatine phosphate. Phosphorylation of AMPK by an upstream kinase (AMPKK), which is also activated by 5′-AMP, also results in activation. A large amplification of activity can result from maximal stimulation by both mechanisms. Hardie, D. G. & D. Carling Eur. J. Biochem. 246:259-273, 1997; Hardie, D. G., et al. Annu. Rev. Biochem. 67:821-855, 1998; Winder, W. W., & D. G. Hardie, Am. J. Physiol. 277:E1-10, 1999. The AMPK activity increases in muscle of rats running on the treadmill, and in response to electrical stimulation. Ruderman, N. B., et al. Am. J. Physiol. 276:E1-E18, 1999; Winder, W. W., & D. G. Hardie Am. J. Physiol. 270:E299-E304, 1996; Winder, W. W., & D. G. Hardie, Am. J. Physiol. 277:E1-10, 1999. However, it has not been determined whether the chronic chemical activation of AMPK will increase mitochondrial enzymes, GLUT4, and hexokinase in different types of skeletal muscle of a resting mammal.

In light of the foregoing, it would be an advancement in the art to provide a method of treating obesity that stimulates a pathway that responds to exercise training. It would be a further advancement to provide a method that increases mitochondrial enzymes in an obese person. It would be an additional advancement if the method reduced the body fat percentage of a subject. It would be a further advancement to provide a method that could mimic exercise training for an extended period of time. Such methods are disclosed and claimed herein.

### 4. BRIEF SUMMARY OF THE INVENTION

The invention relates to a method of treating obesity in a mammal. The method includes the step of administering a therapeutically effective amount of an AMP-activated protein kinase activator to the mammal. The mammal may be for example, a human, a rat, a mouse, and the like. The AMP-activated protein kinase activator can be subcutaneously injected into the mammal or administered in any other manner that provides for uptake of the AMP-activated protein kinase activator into the cells of the mammal. The activation of the AMP-activated protein kinase activator can produce the benefits of exercise training including the loss of body fat.

The invention also relates to a method of treating insulin resistance in a mammal suffering from obesity, type 2 diabetes, or muscle paralysis. To reduce the insulin resistance a therapeutically effective amount of an AMP-activated protein kinase activator is given to the mammal. An AMP-activated protein kinase activator can also be used as an ergogenic aid to treat for example, paralyzed muscle or to enhance the mitochondrial eznyme content in muscle of athletes, armed forces personnel, and others where an increased mitochondrial content of muscle would be advantageous. Another possible use may be for treatment of individuals requiring prolonged bed rest or inactivity which have been shown to cause regression in muscle mitochondrial content.

AMP-activated protein kinase can be activated allosterically by increases in the concentration of AMP or by a compound that is analogous to AMP. In one aspect of the invention an AMP analog such as adenosine-5′-thiomonophosphate, adenosine 5′-phosphoramidate, formycin A 5′-monophosphate, or ZMP is administered to a subject so that the AMP analog is taken into the cells of the subject. This may require modification of the analog so that it may be transported into the cell. Because these AMP analogs are not readily transported into a cell the analog may be administered intracellularly.

5-aminoimidazole-4-carboxamide ribonucleoside (AICAR) is an adenosine analog that is phosphorylated in muscle cells to become ZMP. This allows the 5-aminoimidazole-4-carboxamide to enter the cells and then be converted to ZMP to mimic the effect of AMP in the cell. 5-aminoimidazole-4-carboxamide ribonucleoside can be administered at a dose from about 0.5 to at least about 1.0 mg/g body weight.

In one aspect of the invention, the AMP-activated protein kinase activator is administered acutely in a single dose. The AMP-activated protein kinase activator is administered chronically over a period of weeks to provide an additional benefit to the subject. To better mimic the effect of exercise training the AMP-activated protein kinase activator can be administered intermittently for a period of time.

### 5. SUMMARY OF THE DRAWINGS

A more particular description of the invention briefly described above will be rendered by reference to the appended drawings and graphs. These drawings and graphs only provide information concerning typical embodiments of the invention and are not therefore to be considered limiting of its scope.

FIG. 1 is a set of graphs illustrating the food intake and body weight of rats injected with AICAR.

FIG. 2 is a set of bar graphs illustrating δ-Aminolevulinate synthase and cytochrome c protein expression in rats injected with AICAR.

FIG. 3 is a bar graph illustrating the citrate synthase activity in rats injected with AICAR or saline.

FIG. 4 is a set of bar graphs illustrating succinate dehydrogenase and malate dehydrogenase in rats injected with AICAR or saline.

FIG. 5 is a set of bar graphs illustrating hexokinase activity and GLUT4 in rats injected with AICAR.

FIG. 6 is a schematic representation of the putative actions of AMPK in skeletal muscle.

FIG. 7 is a bar graph illustrating the AMPK activity in gastrocnemius muscles from denervated rats acutely treated with saline or AICAR.

FIG. 8 is a bar graph illustrating the AMPK activity in soleus muscles from denervated rats acutely treated with saline or AICAR.

FIG. 9 is a bar graph illustrating the ACC specific activity for soleus muscles from denervated rats treated with saline or AICAR.

FIG. 10 is a graph showing citrate dependence of acetyl-CoA carboxylase (ACC) in denervated and contralateral innervated gastrocnemius muscles from rats treated with AICAR.

FIG. 11 is a graph showing dose dependent response of GLUT4 levels.

FIG. 12A is a bar graph showing GLUT4 protein content in gastrocnemius muscles.

FIG. 12B illustrates a Western Blot showing GLUT4 protein content in gastrocnemius muscles.

FIG. 13 is a bar graph showing the GLUT4 protein concentration in soleus muscles from saline and AICAR treated rats.

### 6. DETAILED DESCRIPTION OF THE INVENTION

The invention relates to a method of treating obesity in a mammal. The method of the present invention may also be used to treat insulin resistance in a mammal suffering from obesisty, type 2 diabetes, or muscle paralysis. The present invention also relates to ergogenic aids that can enhance the building of muscle in response to exercise.

The method includes the step of administering a therapeutically effective amount of an AMP-activated protein kinase activator to the mammal. The term therapeutically effective amount as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated. In other words, therapeutically effective amount is intended to mean an amount of a compound sufficient to produce the desired pharmacological effect. It is understood that the therapeutically effective amount to be used in the treatment of obesity, insulin resistance, or as an ergogenic aid must be subjectively determined according to the type of mammal and the desired effect. Variables involved include the size of the patient, the type of AMPK activator, the state of the disease, age of the patient, and response pattern of the patient. The novel methods of the invention for treating, preventing or alleviating the conditions described herein, comprise administering to mammals in need thereof, including humans, an effective amount of one or more compounds of this invention or a non-toxic, pharmaceutically acceptable addition salt thereof. The compounds may be administered subcutaneously, orally, rectally, parenterally, or topically to the skin and mucosa. Moreover, because many of the known AMP analogs are phosphorylated, it is difficult to get an effective amount of the analog inside a cell by injection or topical methods. Thus, it may be necessary to administer the analog directly into the muscle of the mammal by for example methods of in vivo electroporation.

The mammal may be for example, a human, a rat, a mouse, and the like. The AMP-activated protein kinase activator can be subcutaneously injected into the mammal or administered in any other manner that provides for uptake of the AMP-activated protein kinase activator into the cells of the mammal. The activation of the AMP-activated protein kinase activator can produce the benefits of exercise training including the loss of body fat.

AMP-activated protein kinase can be activated allosterically by increases in the concentration of AMP or by a compound that is analogous to AMP. In one aspect of the invention an AMP analog such as adenosine-5′-thiomonophosphate, adenosine 5′-phosphoramidate, formycin A 5′-monophosphate, or ZMP is administered to a subject so that the AMP analog is taken into the cells of the subject. This may require modification of the analog so that it may be transported into the cell. Because these AMP analogs are not readily transported into a cell the analog may be administered intracellularly.

5-aminoimidazole-4-carboxamide ribonucleoside (AICAR) is an adenosine analog that is phosphorylated in muscle cells to become ZMP. This allows the 5-aminoimidazole-4-carboxamide to enter the cells and then be converted to ZMP to mimic the effect of AMP in the cell. 5-aminoimidazole-4-carboxamide ribonucleoside can be administered at a dose from about 0.5 to at least about 1.0 mg/g body weight. It has been shown that an effective dose in a rat is about 1.0 mg/g body weight. However, when determining the dose the treatment of a human, the dose may be higher or lower.

In one aspect of the invention, the AMP-activated protein kinase activator is administered acutely in a single dose. This provides the acute activation of AMPK and provides for a short-lived effect similar to exercising once. However, the AMP-activated protein kinase activator can be administered chronically over a period days or weeks to provide an additional benefit to the subject. Providing a dose for a chronic period of about 28 days has been shown to give significant benefits over a single acute activation of AMPK.

The AMPK activator can also be administered intermittently over a period of time to better mimic the effect of exercise training. Such intermittent activation can consist of activating AMPK for a period of at least one day, followed by a period of non-activation for at least one day, followed by an additional period of activation of at least one day. The period of activation followed by non-activation can be repeated as needed to obtain the desired results. For example an increased effectiveness was observed when rats were intermittently injected with AICAR as follows: injection for 3 days, followed by 2 days without injection, followed by 5 days of injection, followed by two days without injection, followed by 3 days with injection.

5′-aminoimidazole-4-carboxamide 1-β-D-ribofuranoside, also known as AICA riboside and AICAR, is an analogue of adenosine that is taken up into the cell and phosphorylated to form ZMP, an analog of 5′-Adenosine monophosphate (AMP). During contraction, free AMP accumulates in the muscle fibers and activates an enzyme, AMP-activated protein kinase (AMPK), which in turn phosphorylates specific enzymes involved in fat oxidation and glucose uptake. AMPK phosphorylates muscle acetyl-CoA carboxylase, thereby decreasing its activity and reducing the production of malonyl-CoA, an inhibitor of fatty acid oxidation in muscle. Artificial activation of AMPK in perfused rat hindlimbs with AICA riboside stimulates an increase in fatty acid oxidation and an increase in glucose uptake. Rats have been chronically treated with subcutaneous injections of AICA-riboside for 4 weeks. This treatment was found to artificially activate AMPK and reduce acetyl-CoA carboxylase activity and malonyl-CoA in muscle of resting rats. These changes would be expected to cause an increase in the rate of fatty acid oxidation by muscle. These rats had significant reductions in the size of the fat pads compared to pair fed control rats. Prolonged treatment of rats with AICAR results in increases in the amount of GLUT4 (the insulin-sensitive glucose transporter) and hexokinase, proteins which can influence the degree of insulin sensitivity. Both of these actions would be beneficial for patients who are obese. AMPK activators, such as AICA riboside, would be useful for treatment of obesity and of the insulin resistance associated with obesity.

It has long been known that regular endurance exercise (1-2 hr of running, swimming, or cycling) over a period of days, weeks and months, increases mitochondria, increases the amount of insulin-sensitive glucose transporter protein (GLUT4) in muscle, and increases insulin sensitivity. Conclusive data is provided showing that chronic chemical activation of the AMP-activated protein kinase with AICA riboside results in an increase in the total quantity of GLUT4 and mitochondrial enzymes in the muscle of rats. This chronic chemical activation of AMPK may prove useful in increasing insulin-sensitivity of type 2 diabetics who can not exercise, for increasing insulin sensitivity of muscle in patients confined to bed rest, and for increasing GLUT4, insulin sensitivity and muscle mitochondrial levels in individuals with muscle paralysis. It also may be useful as an ergogenic aid, producing an accelerated training effect (ie increase in mitochondrial oxidative enzymes, GLUT4 and hexokinase) on muscle.

AMP-activated protein kinase activators may be used to treat insulin resistance in type 2 diabetes and in individuals with muscle paralysis. AMP-activated protein kinase activators such as AICA-riboside, may also be used as ergogenic aids, producing many of the intramuscular adaptations that occur with prolonged endurance exercise training.

Other analogues of 5′-AMP have been found to activate AMPK more potently in vitro including adenosine-5′-thiomonophosphate and adenosine 5′-phosphoramidate. Formycin A 5′-monophosphate and ZMP activate less potently. See Hardie & Carling, Eur. J. Biochem. 246:259-273 (1997). AICA-riboside is the only adenosine analog that has been found useful in activating AMPK in vivo and which has been utilized to show effects of chronic activation of this kinase.

In acute studies rats were injected subcutaneously with 5-aminoimidazole-4-carboxamide-1-β-D-ribofuranoside (AICAR) (1 mg/g b.w.) in 0.9% NaCl or with 0.9% NaCl alone and then were anesthetized for collection and freezing of tissues. AMPK activity increased in the superficial white region of the quadriceps and in soleus muscles but not the deep red region of the quadriceps muscle. Acetyl-CoA carboxylase activity (ACC), a target for AMPK, decreased in all three muscle types, but was lowest in the white quadriceps in response to AICAR injection. In rats given daily subcutaneous injections of AICAR (1 mg/g) for 4 wk, activities of citrate synthase, succinate dehydrogenase and malate dehydrogenase were all increased in white quadriceps and soleus but not in red quadriceps. Cytochrome c and delta-aminolevulinic acid synthase were increased in white but not in red quadriceps. Carnitine palmitoyl-transferase and hydroxy-acylCoA dehydrogenase were not significantly increased. Hexokinase was markedly increased in all three muscles and GLUT4 was increased in red and white quadriceps. These results suggest that chronic AMPK activation may mediate the effects of muscle contraction on some but not all of the biochemical adaptations of muscle to endurance exercise training.

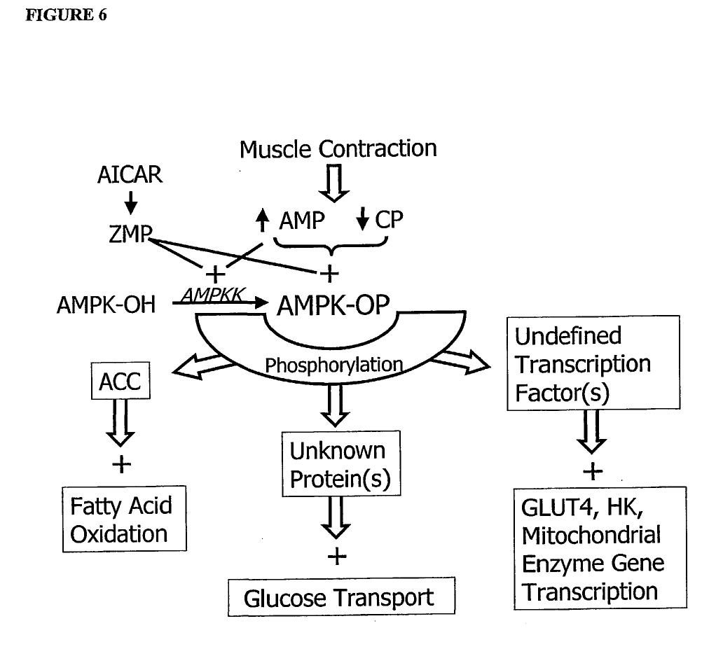

Referring to FIG. 6, the putative actions of AMPK in skeletal muscle is illustrated. Effects on fatty acid oxidation, glucose transport, and expression of GLUT4 and hexokinase (HK) have been described previously. Holmes, B. F., et al. J. Appl. Physiol. 87:1990-1995, 1999; Winder, W. W., & D. G. Hardie, Am. J. Physiol. 277:E1-10, 1999. Effects on mitochondrial enzymes are reported herein. Of these three actions, acetyl-CoA carboxylase (ACC) is the only phosphorylation target for AMPK that has been identified in skeletal muscle. AMPK is naturally activated during muscle contraction, but may be activated artificially by injecting AICAR subcutaneously into rats or by exposing incubated or perfused muscle to AICAR. AMP may directly activate AMPK allosterically or may activate the AMPKK (allosterically) which in turn phosphorylates/activates AMPK. Creatine Phosphate (CP) is an allosteric inhibitor of AMPK. The decline in CP during muscle contraction is thought to relieve inhibition of AMPK, resulting in increased activity.

It was determined if mitochondrial adaptations could also be mediated by chronic AMPK activation. ZMP is increased in muscle 15 minutes following a subcutaneous injection of AICAR and remains elevated for at least two hours following the injection. It was found that AMPK activity was increased in white quadriceps and soleus, but not in red quadriceps 60 minutes following injection of the AICAR. It should be emphasized that AMPK can be activated by phosphorylation by an upstream kinase (AMPKK) and also by allosteric mechanisms (FIG. 6). The AMPK activity that was measured in muscle extracts provides an indirect measure of the phosphorylation state of AMPK. However, both AMPKK and AMPK are also activated allosterically by the free AMP concentration, which has been shown to increase in the muscle in response to contraction. Winder, W. W., & D. G. Hardie, Am. J. Physiol. 277:E1-10, 1999. In addition, AMPK is inhibited allosterically by creatine phosphate, making the decline in CP during muscle contraction an important signal for allowing activation of this enzyme. Winder, W. W., & D. G. Hardie, Am. J. Physiol. 277:E1-10, 1999. These allosteric effects are lost during preparation of the AMPK extracts and can not therefore be detected with the AMPK assay. However, it is possible to obtain an estimation of the in vivo activation of AMPK by measuring the effects of phosphorylation of one of its known target proteins.

Purified muscle acetyl-CoA carboxylase (ACC) has been shown to be phosphorylated in vitro by AMPK. Winder, W. W., & D. G. Hardie Am. J. Physiol. 270:E299-E304, 1996. This also results in an increase in the activation constant for citrate (Ka) and a decrease in maximal velocity (Vmax) of the purified ACC. Winder, W. W., & D. G. Hardie Am. J. Physiol. 270:E299-E304, 1996. Similar changes in kinetic properties of ACC are observed in muscle of rats running on the treadmill and in electrically stimulated muscle. Ruderman, N. B., et al. Am. J. Physiol. 276:E1-E18, 1999; Winder, W. W., & D. G. Hardie Am. J. Physiol. 270:E299-E304, 1996; Winder, W. W., & D. G. Hardie, Am. J. Physiol. 277:E1-10, 1999. Phosphorylation by AMPK results in marked decreases in activity of ACC at physiological concentrations of citrate (˜0.2 mM). Thus, measurement of ACC activity at 0.2 mM citrate provides information regarding the true activity of AMPK in vivo. Injection of AICAR resulted in decreases in ACC activity in all three muscle types. However, the greatest decrease occurred in the white portion of the quadriceps. The decrease in ACC activity provides evidence that AMPK activity was stimulated in all three muscle types in response to AICAR. In the chronically treated animals and in the red quadriceps prior to chronic treatment, AMPK was apparently activated by predominately allosteric mechanisms (ZMP effects on AMPK) and not by phosphorylation of AMPK by AMPKK.

Citrate synthase, cytochrome c, delta-aminolevulinate synthase (ALA-S), and malate dehydrogenase were all significantly increased in the white region of the quadriceps muscle in response to 4 weeks AICAR injections. The delta-aminolevulinate synthase activity has previously been reported to increase within 16 hours following a prolonged bout of exercise. See Holloszy, J. O., & F. W. Booth. Ann. Rev. Physiol. 38:273-291, 1976. Preliminary experiments on the effect of a single injection of AICAR 16 hours prior to tissue collection have produced negative results, however (data not shown). Hexokinase activity increased in response to AICAR in all muscle types. Interestingly, neither of the enzymes involved in fatty acid oxidation that was measured increased in response to AICAR injection. The fact that some enzymes appear to respond and others do not provides evidence that more than one signal is responsible for the concerted rise in mitochondrial oxidative enzyme activity in response to endurance training. It should also be remembered that AICAR injection does not precisely mimic the effects of contraction on this control system. The decline in creatine phosphate accompanying contraction may be a critical component of the signal. Muscle creatine phosphate is not significantly changed in red quadriceps in response to AICAR. The time course of the decline in gastrocnemius ACC activity during electrical stimulation correlates more closely with the decline in creatine phosphate than with the measured AMPK activity (data not shown). The increase in cytosolic calcium during excitation contraction coupling and the rise in plasma fatty acids that accompany prolonged exercise bouts may also be important in inducing the increase in mitochondrial enzymes. These effects of exercise are not mimicked by AICAR injections.

Evidence has been presented recently for a role of calcium and protein kinase C in inducing increases in cytochrome c gene expression. Freyssenet, D., et al. J. Biol. Chem. 274:9305-9311, 1999. An increase in cytochrome c gene expression was observed in myotube culture by treatment with a calcium ionophore. The ionophore-induced response was increased by enhancing expression of calcium-sensitive alpha and beta 2 protein kinase C isoforms, but not of the calcium insensitive delta isoform. It is unclear at this time how the putative AMPK-induced pathway is related to the calcium-triggered pathway. It is also possible that the AMPK pathway is activated with this calcium ionophore. AMPK activity was not quantitated in that study.

It is unclear at this point why the white quadriceps muscle responded with an increase in mitochondrial enzymes and the red quadriceps did not. One possible interpretation is that AMPK activation is not responsible for the mitochondrial adaptations. It is also possible that it is the phosphorylated species of AMPK that is responsible for triggering increased rates of synthesis of mitochondrial enzymes. If the decline in ACC activity is a true measure of in vivo AMPK activity, it might concluded that in red quadriceps AMPK is activated allosterically by AICAR injection, but the phosphorylation state of AMPK is unchanged with respect to controls. It is also important to consider the total daily signal, rather than the isolated effects of AICAR. Certainly the total daily contractile activity would be expected to be greater in red quadriceps and in soleus fibers than in fibers of the white quadriceps.

In addition, the AMPK response to AICAR appeared to be down-regulated by the end of the four week treatment period. No significant increase was observed in AMPK activity in any of the muscle types one hour following injection of AICAR into the chronically-treated rats. The decrease in ACC activity at 0.2 mM citrate was similar however to that seen in rats at the beginning of the four week treatment regimen. The reason for this down-regulation of response is not clear. Significant liver hypertrophy was noted, increasing the probability of more rapid metabolism of the AICAR after the daily injections. However, one hour following injection of the chronically treated rats with AICAR, the ZMP concentration was elevated in all three muscle fiber types to levels in the same range as observed previously. Holmes, B. F., et al. J. Appl. Physiol. 87:1990-1995, 1999. There is also the possibility of a change in expression of the AMPK or AMPI<K genes in response to chronic activation.

When it was apparent that the response of AMPK to AICAR was diminishing over the four week chronic injection of AICAR and that the magnitude of the adaptation appeared to be less than that seen in response to endurance training 2 hr/day (2 fold increase), it seemed important to determine if an intermittent pattern of AICAR injection would prevent down regulation, allowing the true response of a mitochondrial marker enzyme to AMPK activation to be observed. Previous studies clearly demonstrated that training adaptations occur in rats run only five days/week, 2 hr/day, representing an intermittent stimulus. Holloszy, J. O. J. Biol. Chem. 242:2278-2282, 1967. The fact that a significant increase in citrate synthase was observed in red quadriceps in response to two weeks of intermittent treatment with AICAR suggests the possibility that in the four week chronic injection study, the mitochondrial enzymes may have increased earlier in the treatment, but because of down regulation, subsided in red quadriceps as the treatment was extended to four weeks.

The marked decrease in fat pad size is of considerable interest in terms of treatment of type 2 diabetes and obesity using AMPK activators. The chronic AMPK activation is accompanied by ACC inactivation and a consequent decline in malonyl-CoA. See Holmes, B. F., et al. J. Appl. Physiol. 87:1990-1995, 1999. The decline in malonyl-CoA would allow an increase in flux of fatty acids into the mitochondria and an increase in fatty acid oxidation. AICAR-treated rats were pair-fed so that they ate the same amount of food and gained weight at the same rate as controls, but the size of the fat pads was still markedly decreased. These results provide an additional rationale for development of more potent AMPK activators for treatment of type 2 diabetes and obesity. The large amount of AICAR required to induce these adaptations make it an unlikely candidate for use in human patients for these purposes.

Insulin resistance has been observed in the denervated muscles of rats three days after denervation. This resistance has been partially explained by noting the high correlation between decreased insulin-stimulated glucose uptake and the decrease of total GLUT4 in muscle cells. Megeney L A, et al. Am J. Physiol 264 (Endocrinol Metab 27): E583-E593, 1993; Zhou M, et al. Am J. Physiol 278 (Endocrinol Metab 6): E1019-E1026, 2000. It has also been shown that the insulin signaling pathway is compromised in short and long term denervated muscles. Hirose M, et al. Metabolism 50(2):216-22, 2001; Turinsky J & Damrau-Abney A, Am J. Physiol 275 (Regulatory Integrative Comp Physiol 44): R1425-R1430, 1998; Wilkes J J & Bonen A, Am J. Physiol Endocrinol Metab 279: E912-E919, 2000. Further, Han et al have concluded that the reductions in contraction-stimulated glucose transport of denervated muscles is attributable to reductions in GLUT4 and not to lack of stimulation or loss of muscle force production. Han X-X, et al. Mol Cell Biochem 210: 81-89, 2000. Since it is know that contraction stimulates glucose uptake via a pathway different from insulin, and since it has also been convincingly demonstrated that AMPK is an important intermediate regulator in the pathway that stimulates glucose uptake via contraction, the fact that chemical activation of AMPK in muscle cells can be accomplished via subcutaneous injection of AICAR provides interesting possibilities for treatment of such glucose transport impairments that affect the insulin but not the contraction GLUT4 signaling pathway. Goodyear L J, et al. Am J. Physiol 268:E987-E995, 1995; Hayashi T, et al. Am J. Physiol 273: E1039-E1051, 1997; Lund S, et al. Proc Natl Acad Sci 92: 5817-5821, 1995; Hayashi T, et al. Diabetes 47: 1369-1373. 1998; Holmes B F, et al. J. Appl Physiol 87(5):1990-1995, 1999; Kurth-Kraczek E J, et al. Diabetes 48: 1667-1671, 1999; Merrill G F, et al. Am J. Physiol 273 (Endocrinol Metab 36): E1107-E1112, 1997. While previous studies using perfused hindlimbs, isolated epitrochlearis muscles, and live rats have shown that acute AMPK activation via AICAR administration effects an increase in glucose uptake, in this study the possibility of increasing GLUT4 expression by chemically activating AMPK in denervated muscles was examined. Kurth-Kraczek E J, et al. Diabetes 48: 1667-1671, 1999; Merrill G F, et al. Am J. Physiol 273 (Endocrinol Metab 36):E1107-E1112, 1997; Hayashi T, et al. Diabetes 47: 1369-1373. 1998; Bergeron, R, et al. Am J. Physiol 276 (Endocrinol Metab 39): E938-E944, 1999; Buhl E S, et al. Diabetes 50:12-17, 2001.

AMPK activity is markedly increased in denervated gastrocnemius muscles but not in denervated soleus muscles by acute injectin of AICAR. Further, AMPK activity is significantly increased in contralateral innervated AICAR-treated gastrocnemius and soleus muscles. ACC activity is used in this investigation as a reporter enzyme to indicate the in vivo action of AMPK since the AMPK activity assay is performed on ammonium sulfate precipitated homogenates that do not reflect any modulation of AMPK activity due to allosteric effects. ACC activity is markedly decreased in the denervated and contralateral innervated AICAR-treated gastrocnemius and soleus muscles. The increase in AMPK activity in AICAR-treated denervated muscles is highlighted by the changes in kinetic properties for ACC activation in this study which mirror the changes in the kinetic properties for ACC which has been purified and phosphorylated in vitro. Winder WW & Hardie D G, Am J. Physiol 270 (Endocrinol Metab 33): E299-E304, 1996. In muscle cells, activation of AMPK is associated with a decrease in ACC activity. Henin N, et al. FASEB J9(7): 541-546, 1995; Vavvas D, et al. J. Biol Chem 282:13256-13261, 1997; Winder W W & Hardie D G, Am J. Physiol 270 (Endocrinol Metab 33): E299-E304, 1996. This inhibitory influence of AMPK on ACC is significant because the product of ACC, malonyl CoA, is a powerful inhibitor of carnitine palmitoyl-transferase 1 (CPT-1), the enzyme responsible for allowing the passage of fatty acids into the mitochondria. So, increased AMPK activity, besides being a regulatory enzyme in the pathway for contraction-mediated glucose transport, is also associated with increased fatty acid oxidation. See Winder W W & Hardie D G, Am J. Physiol 277 (Endocrinol Metab 40): E1-E10, 1999. It follows then that AMPK activation via AICAR injection in denervated muscles might also provide a way to regulate fatty acid oxidation in denervated muscles.

Like previous studies examining GLUT4 content in denervated muscles, it was found that GLUT4 was significantly decreased in gastrocnemius muscles and soleus muscles 3 days after denervation. Block N, et al. J. Clin Invest 88:1546-1552, 1991; Castello A, et al. J. Biol Chem 268:14998-15003, 1993; Han X-X, et al. Mol Cell Biochem 210: 81-89, 2000; Henriksen I J, et al. J. Appl Physiol 70: 2322-2327.1991; Megeney L A, et al. Am J. Physiol 264 (Endocrinol Metab 27): E583-E593, 1993. Subcutaneous AICAR injections resulted in 80% increase of GLUT4 in denervated gastrocnemius muscles vs. saline-treated denervated gastrocnemius controls. Soleus muscles of AICAR-treated rats, which exhibited a significant decrease in ACC activity but did not exhibit a significant increase in AMPK activity vs. saline-treated controls, did not manifest a significant increase in GLUT4. This might suggest that the phosphorylated form of AMPK is active in the ACC activation pathway but that the allosterically-activated form of AMPK is active in the pathway responsible for increasing GLUT4 expression. This result is consistent with a previous study which suggests that increased AMPK activity induced by AICAR injection effects increases in total GLUT4 content in a fiber type-specific manner with the greatest effect being seen in white fast-twitch oxidative fibers. Buhl E S, et al. Diabetes 50:12-17, 2001.

There is a correlation between the decrease in GLUT4 and the decrease in insulin-stimulated glucose uptake (r=0.99) in denverated muscle. Megeney L A, et al. Am J. Physiol 264 (Endocrinol Metab 27): E583-E593, 1993. In support of the idea that loss of GLUT4 is responsible for reduced insulin-stimulated glucose uptake in 3 day denervated muscles, a recent investigation examined the translocation of elements known to co-localize with GLUT4 in intracellular compartments and translocate in response to insulin. It was shown that there was no difference in the insulin-stimulated translocation of insulin-responsive aminopeptidase (IRAP), transferrin, and insulin-like growth factor II/mannose 6-phosphate in denervated vs. control EDL muscles. Zhou M, et al. Am J. Physiol 278 (Endocrinol Metab 6): E1019-E1026, 2000. It is not the insulin signaling pathway leading to GLUT4 translocation that is impaired, rather that it is the suppressed expression of GLUT4 that limits insulin-stimulated glucose transport in denervated hindlimbs.

IRS-1 phosphorylation in the tibialis anterior, P13-K activity in the tibialis anterior, and Akt-1 kinase activity in soleus and plantaris muscles are all significantly decreased in long term denervated muscles when stimulated by insulin. Hirose M, et al. Metabolism 50(2):216-22, 2001; Turinsky J & Damrau-Abney A, Am J. Physiol 275 (Regulatory Integrative Comp Physiol 44): R1425-R1430, 1998. This suggests that beyond the loss of total GLUT4 there is another level of regulation that is responsible for the decrease in insulin-stimulated glucose uptake. Since it is possible to reverse the depletion of total GLUT4 in certain 3 day denervated muscles, it will be possible to shed new light on the question of insulin-stimulated glucose uptake in denervated muscles.

It is possible to increase GLUT4 levels in paralysis patients with spinal cord injury via electrical stimulation exercise over an 8-week period. Chilibeck P D, et al. Metabolism 48: 1409-1413, 1999; Hjeltnes N, et al. FASEB J. 12:1701-1712, 1998. The congruous increases in GLUT4 that was found by chemically activating AMPK in denervated muscles provide basis for the possibility of pharmacologically manipulating the glucose utilization system in such patients.

In order to better describe the details of the present invention, the following discussion is divided into seven sections: (1) GLUT4 translocates from microvesicles in response to insulin and contraction; (2) AICAR is an adenosine analogue; (3) denervation of rat hind limbs; (4) activation of AMPK; (5) animal care; (6) western blot analysis for GLUT4; and (7) statistical analysis.

6.1 GLUT4 Translocates from Microvesicles in Response to Insulin and Contraction

In muscle, GLUT4 translocates from microvesicles to the sarcolemmal and T-tubular membranes in response to insulin and contraction. See Pessin J E, et al. J. Biol Chem 274:2593-2596, 1999; Goodyear L J, & Kahn B B. Ann Rev Med 49:235-261, 1998; Hayashi T, et al. Am J. Physiol 273: E1039-E051, 1997. GLUT4 translocation stimulated by contraction uses a separate pathway than does insulin. Goodyear L J, et al. Am J. Physiol 268: E987-E995, 1995; Hayashi T, et al. Diabetes 47: 1369-1373. 1998; Hayashi T, et al. Am J. Physiol 273: E1039-E1051, 1997; Lund S, et al. Proc Natl Acad Sci 92: 5817-5821, 1995. Recent studies have shown that the contraction-mediated pathway is dependent on 5′-AMP-activated protein kinase (AMPK). Hayashi T, et al. Diabetes 47: 1369-1373. 1998; Kurth-Kraczek E J, et al. Diabetes 48: 1667-1671, 1999.

6.2 AICAR Is an Adenosine Analogue

5-aminoimidazole-4-carboxamide-riboside (AICAR) is an adenosine analogue that is phosphorylated in muscle cells to become ZMP. Chemically induced activation of AMPK via AICAR has been shown to stimulate insulin-stimulated glucose uptake in live rats, appearance of GLUT4 at the cell membrane in live rats, GLUT4 translocation in perfused hindlimbs, and glucose uptake in isolated epitrochlearis muscles via the same pathway as contraction. Bergeron, R, et al. Am J. Physiol 276 (Endocrinol Metab 39): E938-E944, 1999; Buhl E S, et al. Diabetes 50:12-17, 2001; Kurth-Kraczek E J, et al. Diabetes 48: 1667-1671, 1999; Merrill G F, et al. Am J. Physiol 273 (Endocrinol Metab 36): E1107-E1112, 1997; Hayashi T, et al. Diabetes 47: 1369-1373. 1998. Furthermore, while it has long been known that endurance training increases GLUT4 expression in animals and in humans, more recent studies have demonstrated that increased AMPK activity is essential in mediating this increase in GLUT4 expression in these chronically contracting muscles. Goodyear L J, et al. Diabetes 41:1091-1099, 1992; Ploug T, et al. Am J. Physiol 259 (Endocrinol Metab 22): E778-E786, 1990; Rodnick K J, et al. Diabetes 39:1425-1429, 1990; Dela F, et al. Diabetes 43:862-865, 1994; Houmard J A, et al. Am J. Physiol 261 (Endocrinol Metab 24): E437-E443, 1991; Houmard J A, et al. Am J. Physiol 264 (Endocrinol Metab 27): E896-E901, 1993; Hughes V A, Fiatarone M A, Fielding R A, Kahn B B, Ferrara, C M, Shepherd P, Fisher E C, et al. Am J. Physiol 264 (Endocrinol Metab 27): E855-E862, 1993; Holmes B F, et al. J. Appl Physiol 87(5):1990-1995, 1999; Ojuka E O, et al. J. Appl Physiol 88:1072-1075, 2000. These same studies have also pointed out the effectiveness of chemically activating AMPK via AICAR in order to mimic the effects of contraction in increasing GLUT4 expression. This study analyzes whether increased AMPK activity induced by subcutaneous injections of AICAR in live rats is effective in increasing the GLUT4 expression in denervated gastrocnemius and soleus muscles.

6.3 Denervation of Rat Hind Limbs

Denervation of rat hindlimbs is a model for glucose transport studies which produces significant muscle atrophy, insulin resistance, and also causes a decrease in the total muscle GLUT4 content. Henriksen I J, et al. J. Appl Physiol 70: 2322-2327. 1991; Burant C F, et al. Am J. Physiol 247: E657-E666, 1984; Turinsky J, Am J. Physiol 252 (Regulatory Integrative Comp Physiol 21): R531-R537, 1987; Block N, et al. J. Clin Invest 88:1546-1552, 1991; Castello A, et al. J. Biol Chem 268:14998-15003, 1993; Coderre L, et al. Endocrinol 131:1821-1825, 1992; Henriksen I J, et al. J. Appl Physiol 70: 2322-2327. 1991. Three days after denervation, when the decrease in GLUT4 is maximal, GLUT4 content in rat hindlimb muscles is significantly decreased as compared to the sham-operated, contralateral control muscles. Block N, et al. J. Clin Invest 88:1546-1552, 1991; Coderre L, et al. Endocrinology 131:1821-1825, 1992; Henriksen I J, et al. J. Appl Physiol 70: 2322-2327. 1991; Kawanaka K, et al. Horm Metab Res 28:75-80, 1996; Megeney L A, et al. Am J. Physiol 264 (Endocrinol Metab 27): E583-E593, 1993.

Denervation also induces a significant decline in mRNA levels in the same three day time period suggesting transcriptional mediation of this change in GLUT4 expression. Block N, et al. J. Clin Invest 88:1546-1552, 1991; Didyk R B, et al. Metabolism 43:1389-1394, 1994; Jones J P, et al. J. Appl Physiol 84(5):1661-1666, 1998. Interestingly, insulin resistance occurs within three hours of denervation and decrease in muscle GLUT4 protein or GLUT4 mRNA content is not observable until two and three days after denervation. Turinsky J, Am J. Physiol 252 (Regulatory Integrative Comp Physiol 21): R531-R537, 1987; Block N, et al. J. Clin Invest 88:1546-1552, 1991; Castello A, et al. J. Biol Chem 268:14998-15003, 1993. Therefore, the insulin resistance associated with 1 day denervation is not wholly due to these changes in GLUT4 content. Yet it is thought that the severity of insulin resistance, which is maximal at 3 days after denervation, is related to the depletion of GLUT4 after 2-3 days. Block N, et al. J. Clin Invest 88:1546-1552, 1991; Coderre L, et al. Endocrinology 131:1821-1825, 1992; Henriksen I J, et al. J. Appl Physiol 70: 2322-2327. 1991. One study even finds an excellent correlation between the decrease in GLUT4 and the decrease in insulin-stimulated glucose uptake (r=0.99) in 3 day denervated muscles. Megeney L A, et al. Am J. Physiol 264 (Endocrinol Metab 27): E583-E593, 1993. Denervation is a useful model for studying glucose transport and insulin resistance because sham-operated, contralateral control muscles are present in the same rat.

Rats were anesthetized with ether and the sciatic nerve of the right hindlimb was severed. The left hindlimb was sham operated to serve as a control. Both skin wounds were closed with a metal clips. The rats used for the acute study were also implanted with a jugular catheter which was exteriorized on the back of the neck at the time of denervation for rapid administration of anesthesia.

6.4 Activation of AMPK

AMPK can be activated allosterically by increases in the concentration of AMP, but is also inhibited by ATP and is therefore sensitive to the AMP/ATP concentration. Corton J M, et al. Current Biol 4:315-324, 1994; Salt IP, et al. Biochem J. 334: 177-187, 1998; Davies SP, et al. FEBS Lett 377:421-425, 1995; Hawley S A, et al. J. Biol Chem 271: 27879-27887, 1996. AMPK is inhibited by creatine phosphate (CP) and is likely sensitive to the CP/creatine (C) ratio. Ponticos M, et al. EMBO J. 17:1688-1699, 1998. AMP also serves to activate an upstream kinase, AMPKK, which in turn phosphorylates and thereby activates AMPK. Davies SP, et al. FEBS Lett 377:421-425, 1995; Hawley S A, et al. J. Biol Chem 270: 27186-27191, 1995. ZMP, a normal intermediate in the purine nucleotide synthetic pathway and an AMP analogue, has been shown to imitate the effects of AMP and stimulate both AMPK and AMPKK. Corton, J M, et al. Eur J. Biochem 229: 558-565, 1995; Henin N, et al. Biochim Biophys Acta 1290:197-203, 1996.

6.5 Animal Care

All procedures were approved by the Institutional Animal Care and Use Committee of Brigham Young University. Male Sprague-Dawley rats (Sasco, Wilmington, Mass.) were housed in individual cages in a temperature- (22-25° C.) and light-controlled (12:12 h light-dark cycle) room and were given Harlan Teklad rodent diet (Madison, Wis.) and water ad libitum. All rats were handled daily for at least 5 days prior to the beginning of treatment to accustom them to the experimental procedures. All rats were approximately 200±30 g. at the beginning of the respective experiments.

6.6 Western Blot Analysis for GLUT4

Muscle was ground to a powder under liquid nitrogen and homogenized (1 mg muscle:9 mL buffer) in HEPES buffer [25 mM HEPES, 1 mM EDTA, 1 mM benzamadine, 1 mM 4-(2-aminoethyl)-benzene+sulfonyl fluoride, 1 μM leupeptin, 1 μM antitrypsin, 1 μM aprotinin, pH 7.5]. This homogenate was then diluted with water and Laemmli's buffer (1 mL homogenate:2 mL water: 1 mL buffer) immediately before loading. 20 μL of this homogenate were then loaded onto a 10% SDS-PAGE mini gel (Tris-HCl ready gels, BioRad, Hercules, Calif.). Samples were subjected to electrophoresis at 200 V for 45 minutes. Proteins were transferred from gel to nitrocellulose membrane at 100V for 50 minutes. Membranes were blocked in 5% non-fat milk (BioRad, Hercules, Calif.) in PBST (139 mM NaCl, 2.7 mM KH₂PO₄, 9.9 mM Na₂HPO₄, and 0.05% Tween-20) and were then left overnight with GLUT4 polyclonal antibody (Biogenesis, Kingston, N.H.) in 5% dried milk solution (1:5000 dilution). After washing twice in PBST, and twice in PBS (139 mM NaCl, 2.7 mM KH₂PO₄, 9.9 mM Na₂HPO₄), the membranes were exposed to horseradish peroxidase-conjugated donkey anti-rabbit IgG (Amersham Life Sciences, Arlington Heights, Ill.) for 1 h. at room temperature. After again washing twice with PBST and twice with PBS, the membranes were incubated in enhanced chemoluminescence-detection reagent and then visualized on enhanced chemoluminescence hyperfilm (Amersham Life Sciences). Relative amounts of GLUT4 were then quantified using a Hewlett Packard Scan Jet 6200C and SigmaGel software (SPSS, Chicago, Ill.). Western blot data are expressed as arbitrary units where individual values were divided by the mean basal GLUT4 value (sham operated leg/injected with 0.9%/NaCl solution).

6.7 Statistical Analysis

Results are expressed as means ±SE. Statistically significant differences between treatment groups were analyzed using Fischer's least significant difference test. Statistical significance is defined as P<0.05.

### 7. EXAMPLES

The following examples are given to illustrate various embodiments which have been made with the present invention. It is to be understood that the following examples are not comprehensive or exhaustive of the many types of embodiments which can be prepared in accordance with the present invention.

### Example 1

### Acute Injection of AICAR

The purpose of these studies was to determine if AMPK activity was acutely increased in muscle by the AICAR treatment. Three days prior to the experiment, jugular cathethers were installed and exteriorized on the back of the neck to allow rapid anesthesia of the rat and rapid blood and tissue collection. Rats were then injected with AICAR (1 mg/g body wt) subcutaneously in sterile 0.9% NaCl or were given 0.9% NaCl (n=6 rats per group). One hour after the subcutaneous injection, rats were anesthetized by intravenous injection of pentobarbital sodium (4.8 mg/100 g body wt). The superficial white and the deep red regions of the quadriceps muscles, and the soleus muscles were quickly removed and frozen with stainless steel clamps at liquid nitrogen temperature.

Resuspended ammonium sulfate precipitates of tissue homogenates were analyzed for AMPK activity and acetyl-CoA carboxylase activity as described previously. Li, B., et al. J. Biol. Chem. 274:17534-17540, 1999; Winder, W. W., & D. G. Hardie Am. J. Physiol. 270:E299-E304, 1996. This measurement of AMPK only detects increases in AMPK activity that survive ammonium sulfate precipitation of the muscle homogenate (ie increases due to phosphorylation) and does not provide information concerning allosteric control by AMP, CP, and ATP. ACC activity at 0.2 mM citrate provides some indication of the in vivo activity of AMPK, since ACC is a target for phosphorylation of AMPK. ACC activity has previously been reported to decrease in response to phosphorylation by AMPK. Winder, W. W., & D. G. Hardie Am. J. Physiol. 270:E299-E304, 1996. The acute experiment was also repeated on rats treated with AICAR or saline for 4 weeks to see if the responses of AMPK and ACC to AICAR persisted for the entire treatment period. In both experiments ATP and CP were measured on neutralized perchloric acid extracts of muscle.

A single injection of AICAR in rats not previously treated with AICAR resulted in significant increases in AMPK activity in white quadriceps and soleus muscle but not in red quadriceps sixty minutes following the injection as seen in Table 1. In these same muscle extracts, the activity of acetyl-CoA carboxylase at 0.2 mM citrate was markedly decreased in all three muscle types. In the AICAR-injected rats, white quadriceps exhibited the lowest activity of ACC (44% of that seen in soleus and 61% of that seen in red quadriceps, p<0.05).

As shown in Table 1, the acute response to AICAR injection was also measured in muscles of rats that had been given daily injections of AICAR for 4 weeks. Table 1 shows the concentrations of ATP (μmol/g), CP (μmol/g), AMP-activated protein kinase (AMPK)(pmol/g/min) and acetyl-CoA carboxylase (ACC) (nmol/g/min at 0.2 mM citrate) in muscles of rats 60 minutes following injection of saline or AICAR. No significant increase in AMPK activity (n=6) was detected in white or red quadriceps or in soleus muscles one hour following injection of AICAR. The ACC activity declined to values similar to those seen in rats prior to chronic AICAR treatment (n=4). The ZMP concentration one hour after injection was 0.42±0.04 μmol/g in white quadriceps, 0.94±0.08 μmol/g in red quadriceps, and 1.15±0.07 μmol/g in soleus muscle. These values were in the same range as those seen in the gastrocnemius muscle 60 min following a single injection of AICAR. See Holmes, B. F., et al. J. Appl. Physiol. 87:1990-1995, 1999. ZMP was not detectable in rats injected with saline. Creatine phosphate was significantly lower in the AICAR injected rats compared to saline-injected controls only in the white quadriceps prior to chronic treatment and in soleus muscle after 4 weeks of AICAR injections. ATP was increased to a small extent by AICAR injection in red quadriceps prior to chronic treatment and in white quadriceps after chronic treatment.

TABLE 1

| White Quadriceps | | Red Quadriceps | | Soleus | |
|---|---|---|---|---|---|
| Control | AICAR | Control | AICAR | Control | AICAR |

Single Injection - No Chronic Treatment

| | | | | | | |
|---|---|---|---|---|---|---|
| CP | 24.6 ± 0.9 | 17.6 ± 0.8* | 25.8 ± 2.8 | 22.4 ± 3.5 | 14.2 ± 0.8 | 12.2 ± 1.2 |
| ATP | 8.2 ± 0.5 | 8.0 ± 0.3 | 8.0 ± 0.4 | 9.2 ± 0.3* | 6.0 ± 0.9 | 6.5 ± 0.8 |
| AMPK | 386 ± 32 | 1,102 ± 80* | 283 ± 30 | 405 ± 62 | 180 ± 17 | 447 ± 42* |
| ACC | 0.75 ± 0.07 | 0.08 ± 0.02* | 0.90 ± 0.06 | 0.19 ± 0.02* | 0.80 ± 0.06 | 0.19 ± 0.02* |

After Daily Injections for 4 Wk

| | | | | | | |
|---|---|---|---|---|---|---|
| CP | 23.8 ± 1.5 | 21.9 ± 0.8 | 21.9 ± 1.3 | 19.6 ± 0.4 | 17.7 ± 0.9 | 13.8 ± 0.9* |
| ATP | 8.3 ± 0.2 | 9.6 ± 0.2* | 7.6 ± 0.2 | 8.6 ± 0.2* | 4.0 ± 0.2 | 5.1 ± 0.5 |
| AMPK | 300 ± 49 | 258 ± 32 | 226 ± 33 | 200 ± 17 | 138 ± 15 | 191 ± 22 |
| ACC | 0.67 ± 0.09 | 0.12 ± 0.01* | 0.72 ± 0.07 | 0.21 ± 0.01* | 0.55 ± 0.06 | 0.28 ± 0.01* |

Values are means ± SEM, n = 4-8 *Significantly different from control value, p <0.05.

### Example 2

### Chronic Injection of AICAR

To determine the effect of chronic injection of AICAR on muscle enyzme activities or expression and on muscle GLUT4, rats were injected subcutaneously (between 8 and 10 a.m.) with AICAR (1 mg/g) or saline vehicle daily for 28±1 days in succession. Beginning with the first injection, controls were pair fed with AICAR-injected rats. Rats were anesthetized by intraperitoneal injection of pentobarbital sodium 22-25 hours after the last AICAR injection and the white and red regions of the quadriceps and the soleus muscles were removed and frozen as described above. Muscles were kept frozen at −70° C. until analyzed. Liver, heart, kidney, and fat pads were weighed.

After obtaining results on blunted acute response to an AICAR injection on AMPK in chronically treated rats, an additional experiment was done to determine if intermittent treatment of rats with AICAR would influence the extent of the increase in citrate synthase. This experiment was also of shorter duration to minimize down regulation that appeared to occur with prolonged treatment. Rats were injected for three days (1 mg/g) followed by two days of no treatment. They were then injected for five days, followed by another two days with no treatment and then for three additional days before killing 20-24 hours following the last injection. Rats were anesthetized and tissues were collected and frozen as before. Analytical methods. Muscles from rats killed 1 h after injection of AICAR or saline were analyzed for AMPK, acetyl-CoA carboxylase (ACC) at 0.2 mM citrate, ATP and CP and ZMP. Li, B., et al. J. Biol. Chem. 274:17534-17540, 1999; Winder, W. W., & D. G. Hardie Am. J. Physiol. 270:E299-E304, 1996; Heinz, F., & H. Weisser. Creatine phosphate. In: Methods of Enzymatic Analysis, edited by H. U. Bergmeyer. New York: Academic, 1983, p. 507-514.

For the chronic effects of AICAR injection muscles were analyzed for glycogen GLUT-4, hexokinase, lactate dehydrogenase, and several mitochondrial enzymes. Hassid, W. Z., & S. Abraham Methods Enzymol. 3:35-36, 1957. GLUT4 was quantitated by western blotting as described previously using GLUT4 polyclonal antibody RaIRGT, Biogenesis, Sandown, N.H.) and horseradish peroxidase-conjugated donkey anti-rabbit IgG (Amersham Life Science, Arlington Heights, Ill.). Holmes, B. F., et al. J. Appl Physiol 87:1990-1995, 1999. δ-Aminolevulinic acid synthase was determined using western blotting techniques as described previously. Li, B., et al. J. Biol. Chem. 274:17534-17540, 1999. For determination of cytochrome c, muscles were homogenized in 10 mM n-2-hydroxyethylpiperazine-N′-2-ethanesulfonic acid (HEPES), 1 mM EDTA, and 250 mM sucrose (HES buffer). Homogenates were centrifuged at 700 g for 10 min and aliquots of the supernatant containing 100 μg protein were solubilized in Laemmli sample buffer, subjected to SDS-polyacrylamide gel electrophoresis (15% resolving gel) and then transferred to nitrocellulose. Cytochrome c was detected by incubating the nitrocellulose blots with a rabbit polyclonal antibody against rabbit heart cytochrome c (Alpha Diagnostics International, San Antonio) followed by horseradish peroxidase-conjugated anti-rabbit immunoglobulin G. Antibody bound GLUT4, δ-Aminolevulinic acid synthase, and cytochrome c were visualized using enhanced chemiluminescence. Protein bands were quantified by densitometry.

For enzyme activity measurements, 10% homogenates were made from the respective muscles in 175 mM KCl, 10 mM GSH, 2 mM EDTA, pH 7.4. This homogenate was frozen and thawed 3 times and mixed thoroughly prior to enzymatic measurements. For succinate dehydrogenase and lactate dehydrogenase, an aliquot of the homogenate was centrifuged at 700×g for 10 min at 4° C. The remainder of the assays were performed on aliquots and dilutions of the mixed whole homogenate. Assays were performed by the following methods: citrate synthase, succinate dehydrogenase, the mitochondrial fraction of malate dehydrogenase, hexokinase, lactate dehydrogenase, carnitine palmitoyl transferase and hydroxyacyl-CoA dehydrogenase. Srere, P. A. Methods in Enzymology 13:3-6, 1969; King, T. E. Methods Enzymol 10:322-335, 1967; Shonk, C. E. & G. E. Boxer, Cancer Res. 24:709-721, 1964; Uyeda, K., & E. Racker, J. Biol Chem 240:4682-4688, 1965; Pesce, A., et al. J. Biol. Chem. 239:1753-1761, 1964; Baldwin, K. M., et al. Am. J. Physiol 222:373-378, 1972; Mole, P. A., et al. J. Clin. Invest. 50:2323-2330, 1971; Kobayashi, A. et al. J. Biochem. 119:775-782, 1996.

Differences between the saline injected control rats and AICAR-injected rats were determined using Student's t test. Values are expressed as means±SEM.

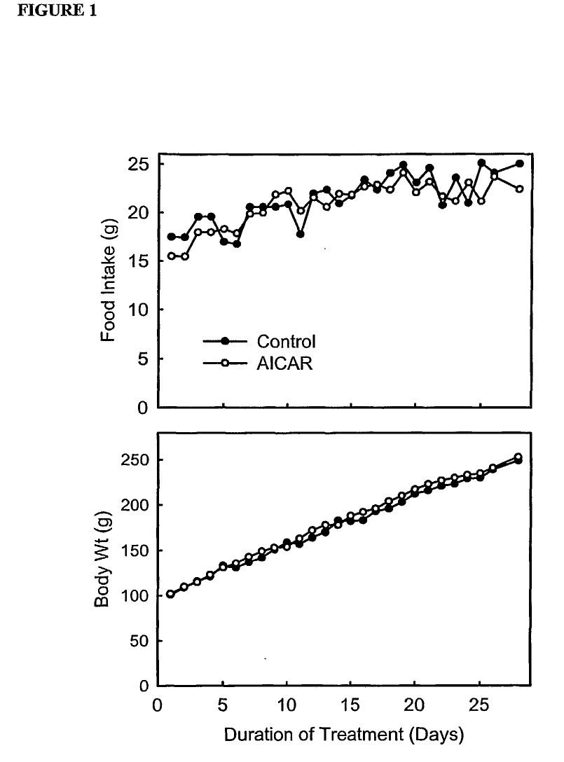

Referring to FIG. 1, food intake and body weight of rats injected with either saline or AICAR in saline for 4 weeks is shown. No statistically significant differences were noted between AICAR and saline injected rats. Final body weights and organ weights are shown in Table 2. No statistically significant differences were noted in muscle, heart, or kidney weights, but the liver showed significant hypertropy in the AICAR-treated animals. There was also a significant decrease in fat pad weight in the AICAR-treated rats.

TABLE 2
Effect of injecting AICAR for four weeks on body
weight (g) and organ weights (g) of rats.

| | Saline Injected Rats | AICAR Injected Rats |
|---|---|---|
| Body Weight | 249 ± 5 | 254 ± 5 |
| Heart | 0.77 ± 0.07 | 0.80 ± 0.02 |
| Gastrocnemius/ | 1.35 ± 0.03 | 1.30 ± 0.04 |
| Plantaris Muscle | | |
| Kidney | 1.02 ± 0.07 | 0.99 ± 0.02 |
| Epididymal Fat Pad | 2.07 ± 0.13 | 1.21 ± 0.10* |
| Retroperitoneal Fat Pad | 1.72 ± 0.18 | 0.90 ± 0.08* |
| Liver | 11.0 ± 0.40 | 16.0 ± 0.50* |

Values are means ± SEM, n = 8. *p < 0.001

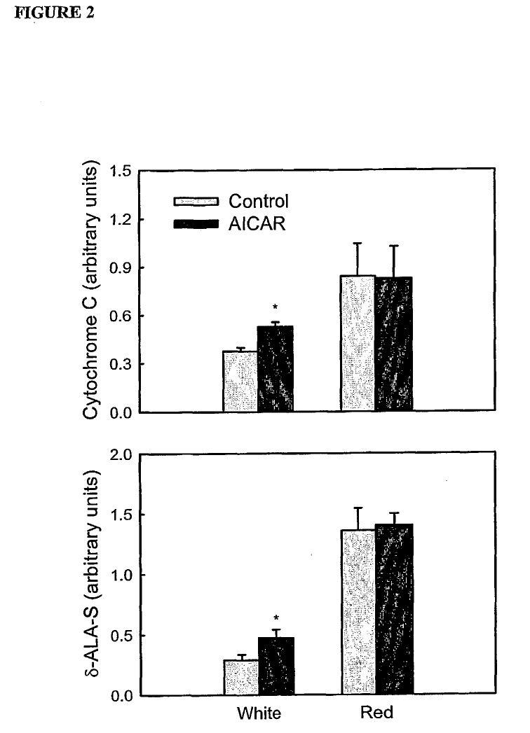

Referring to FIG. 2, the δ-Aminolevulinate synthase and cytochrome c protein expression in red and white portion of quadriceps muscles of rats injected with AICAR or with saline (controls) for 4 weeks is shown. Muscle extracts were subjected to Western blotting using rabbit anti-ALA-S or rabbit antiserum against rabbit heart cytochrome c. Values are means±SEM for 6 muscles per group. An asterisk denotes a significant difference from saline-injected controls, P<0.05. Muscle content of δ-aminolevulinic acid synthase as determined by western blot was significantly increased in white quadriceps, but not in red quadriceps. Cytochrome c was also increased in white but not in red quadriceps.

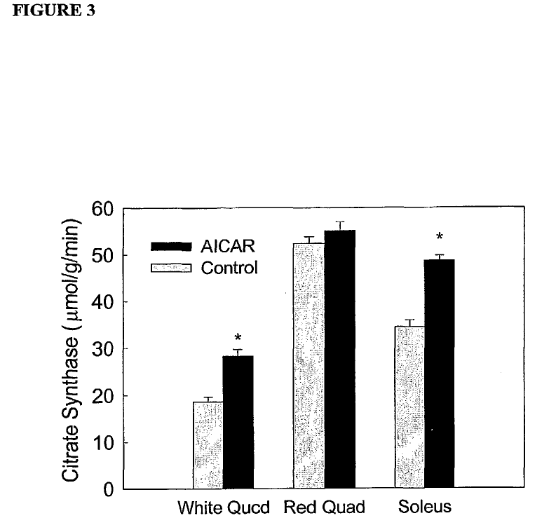

Referring to FIG. 3, the citrate synthase activity in red and white quadriceps and soleus muscles of rats injected with AICAR or saline (controls) for 4 weeks is shown. Values are means±SEM for 8 muscles per group. An asterisk denotes a significant difference from saline injected controls, P<0.001. Citrate synthase was significantly increased in white quadriceps, and soleus, but not in red quadriceps.

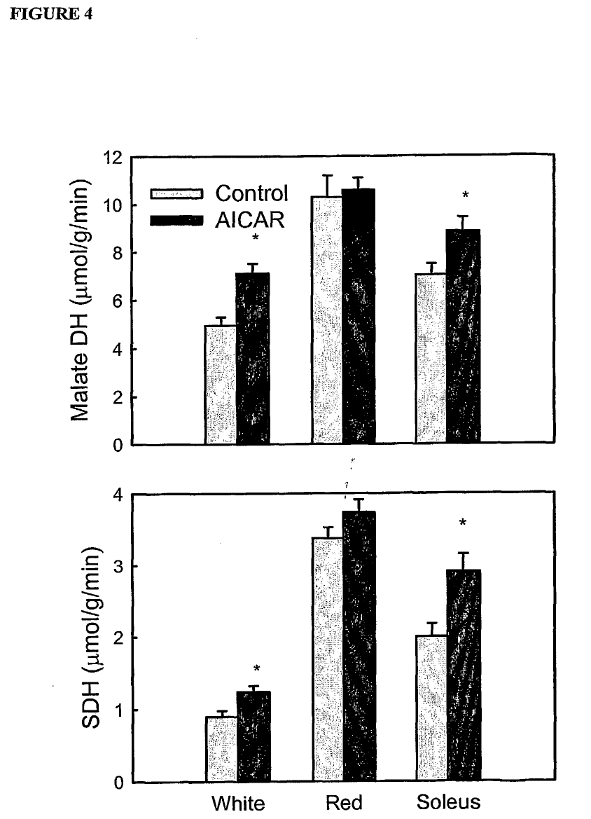

Referring to FIG. 4, the succinate dehydrogenase and malate dehydrogenase activities in red and white quadriceps and soleus muscles of rats injected with AICAR or saline (controls) for 4 weeks are shown. Values are means±SEM for 8-14 muscles per group. These valuse are significantly different from saline injected controls, P<0.05. These two citric acid cycle enzymes, succinate dehydrogenase and malate dehydrogenase showed the same adaptive pattern.

Lactate dehydrogenase, hydroxyacyl-CoA dehydrogenase and carnitine palmitoyl transferase were not significantly influenced in any of the muscle types as shown in Table 3. An increase in glycogen was observed to occur in white and red quadriceps, but not in soleus the day following the last injection of AICAR.

TABLE 3
Effect of chronic AICAR injections on enzyme activities (μmol/g/min)
and glycogen (mg/g) in different types of skeletal muscle.

| | White Quadriceps | | Red Quadriceps | | Soleus | |
|---|---|---|---|---|---|---|
| | Control | AICAR | Control | AICAR | Control | AICAR |
| LDH | 674 ± 15 | 685 ± 20 | 529 ± 14 | 557 ± 26 | 158 ± 11 | 162 ±7 |
| CPT | 0.13 ± 0.01 | 0.16 ± 0.01 | 0.45 ± 0.01 | 0.46 ± 0.02 | 0.55 ± 0.02 | 0.62 ± 0.02 |
| HADH | 3.8 ± 0.4 | 4.6 ± 0.3 | 9.9 ± 0.3 | 9.0 ± 0.4 | 4.2 ± 0.4 | 4.6 ± 0.4 |
| Glycogen | 7.2 ± 0.3 | 12.3 ± 0.4* | 6.9 ± 0.3 | 9.1 ± 0.4* | 6.4 ± 0.4 | 5.8 ± 0.4 |

Values are mean ± SEM, n = 8-14. *p < 0.05. LDH = lactate dehydrogenase, CPT = carnitine palmitoyl-transferase, HADH = hydroxyacyl-CoA dehydrogenase

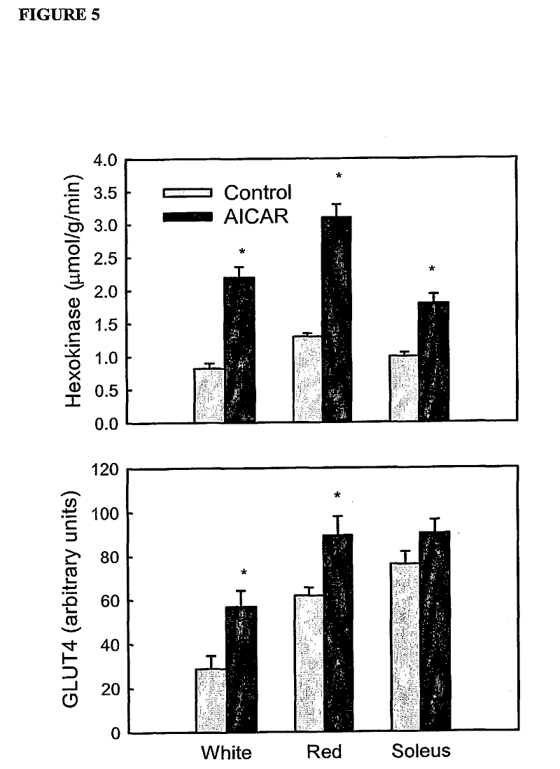

Referring to FIG. 5, the hexokinase activity and GLUT4 (measured by western blot) in the red region of the quadriceps, the white region of the quadriceps, and in soleus of rats injected with AICAR (1 mg/g) for 4 weeks is shown. Values are mean±SEM, n=8. An asterisk denotes a significant difference from saline-injected controls, p<0.05. GLUT4 and hexokinase activity were both significantly increased in response to chronic AICAR injection in red and white regions of the quadriceps. The increase in hexokinase activity in soleus was statistically significant, but the small change in GLUT4 did not reach statistical significance (p=0.12).

In the study with intermittent injections of AICAR, citrate synthase in the white quadriceps was 15.5±1.3 μmol/g/min in controls vs 23.0±1.2 μmol/g/min in AICAR injected rats. This represented a 48% increase and was highly significant (p<0.001). A significant increase also occurred in the red region of the quadriceps (47.0±2.8 μmol/g/min in controls vs 57.4±2.5 μmol/g/min in AICAR injected, p<0.025) in response to AICAR.

### Example 3

### Acute Activation of AMPK in Denervated Rats

AMPK activity was acutely increased in denervated muscles after AICAR injection. Rats were denervated between 8:30 a.m. and 10:30 a.m. and were then given 25 g of food. Rats were handled twice during the day to accustom them to being handled. The rats were given a single subcutaneous injection 24 hours. after surgery of either AICAR (1 mg/g body weight) in sterile 0.9% NaCl or with just 0.9% NaCl, and were subsequently anesthetized intravenously. Soleus and gastrocnemius muscles from both sham and denervated sides were extracted and quick frozen in liquid nitrogen for analysis. AMPK activity and acetyl-Co A carboxylase (ACC) activity were determined as previously described. Winder W W & Hardie D G, Am J. Physiol 270 (Endocrinol Metab 33): E299-E304, 1996. Because this analysis is done on homogenates prepared by ammonium sulfate precipitation, only those effects on AMPK activity modified by phosphorylation are measured. This measurement of AMPK activity does not provide information about how AMPK might be modulated in response to AMP, ATP, or CP allosteric effects. Because ACC is a target for phosphorylation by AMPK, and because ACC activity has been reported to decrease in response to phosphorylation by AMPK, ACC activity is measured to give an indication of the in vivo actions of AMPK.

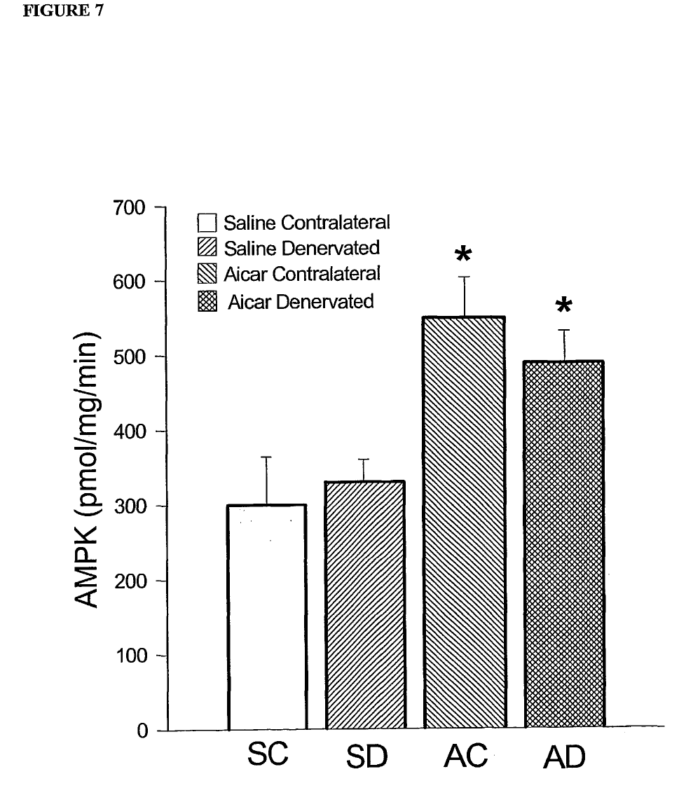

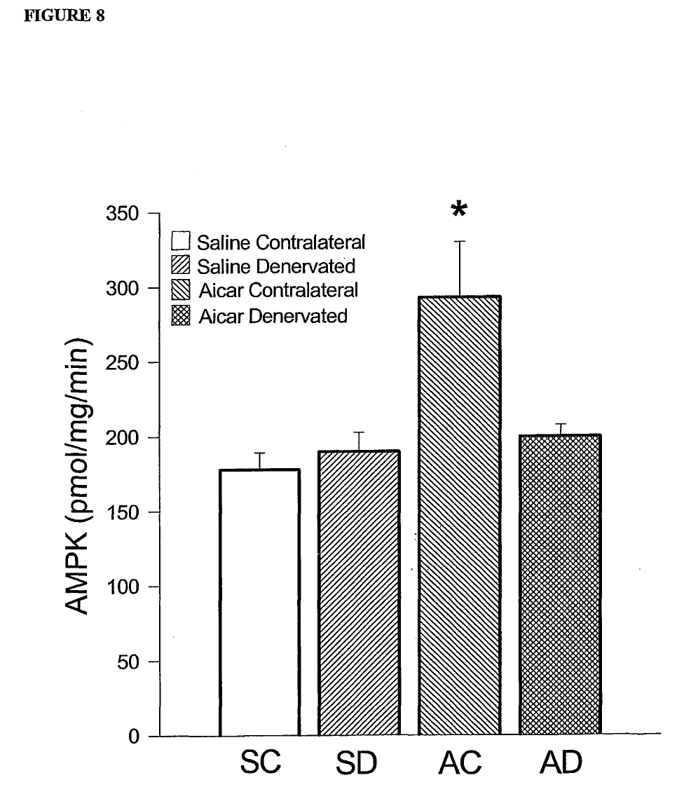

Referring to FIGS. 7 and 8, the first objective was to insure that AMPK was in fact being activated in response to subcutaneous AICAR injections. Using ammonium sulfate precipitated homogenates. In response to an acute injection of AICAR, AMPK activity was significantly (P<0.05) elevated above the control AMPK activity (the AMPK activity in the saline-treated contralateral innervated muscles) in the denervated AICAR-treated gastrocnemius muscles, the contralateral innervated AICAR-treated gastrocnemius, the contralateral innervated AICAR-treated soleus, but not the denervated AICAR-treated soleus (n=4 for each group). FIG. 7 graphs the AMP-activated protein kinase activity in gastrocnemius muscles from denervated rats acutely treated with saline (0.9% NaCl) (n=4 muscles) or with AICAR (1 mg AICAR in 0.9% NaCl/1 g body weight) (n=5 muscles). Values are means±SE. An asterisk indicates P<0.05 vs. saline contralateral and denervated groups. FIG. 8 shows the AMP-activated protein kinase (AMPK) activity in soleus muscles from denervated rats acutely treated with saline (0.9% NaCl) (n=4 muscles) or AICAR (1 mg AICAR in 0.9% NaCl/1 g body weight) (n=5 muscles). An asterisk indicates P<0.05 vs. saline contralateral.

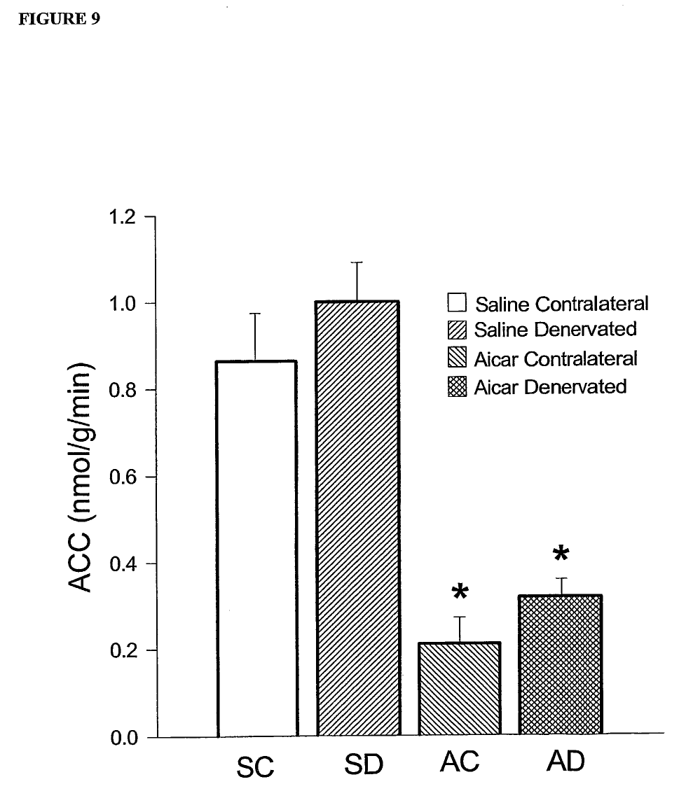

Because the ammonium sulfate precipitated homogenates do not reflect any change in AMPK activity due to allosteric effects, ACC activity was also measured as a demonstration of the in vivo activity of AMPK. While AMPK activity was not found to be significantly increased in AICAR-treated denervated soleus muscles, FIG. 9 shows the ACC specific activity at 0.2 mM citrate for soleus muscle from denervated rats treated with saline (0.9% NaCl) (n=4 muscles) or with AICAR (1 mg AICAR in 0.9% NaCl/1 g body weight) (n=5 muscles). Values are means±SE. An asterisk indicates P<0.01 vs. either of the saline treatment groups. In the physiological range for citrate concentration, ACC activity is significantly (P<0.01) decreased in both the AICAR-treated denervated soleus and the contralateral AICAR treated soleus in comparison to the saline-treated contralateral innervated soleus (n=4 for each group). The collection of ACC activity data from the entire citrate activation curve was not possible due to the limited amount of tissue from soleus muscles.

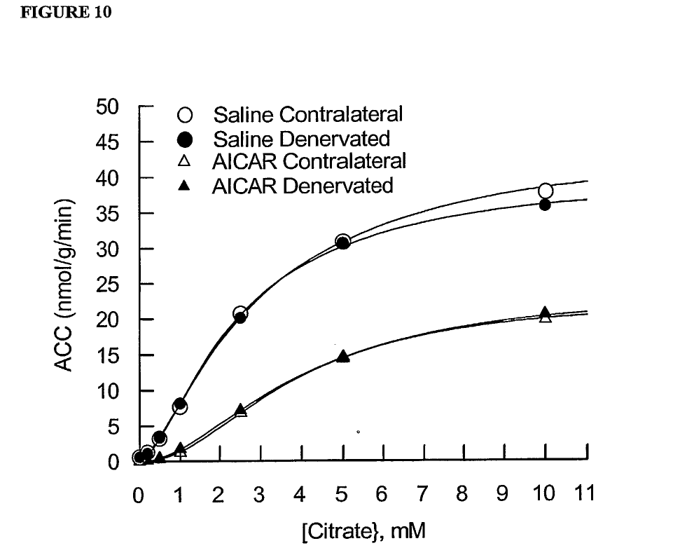

Referring to FIG. 10, Citrate dependence of acetyl-CoA carboxylase (ACC) in denervated and contralateral innervated gastrocnemius muscles from rats treated with AICAR (1 mg AICAR in 0.9% NaCl/1 g body weight) or saline (0.9% NaCl) is illustrated. SE values were determined but are not shown (n=5).Curves were fitted to data by using the Hill equation and Grafit software (Sigma Chemical, St. Louis, Mo.). FIG. 10 graphs the specific activity of ACC in saline-treated and AICAR-treated gastrocnemius muscles at several different citrate concentrations. AICAR injection is shown to significantly decrease ACC activity in both denervated and contralateral innervated gastrocnemius muscles (n=4). The maximal velocity for the reaction (Vₘₐₓ) as a function of increasing citrate concentration is significantly decreased (P<0.01) from 36.7±5 in the saline-treated denervated gastrocnemius to 24.7±1.9 in the AICAR-treated denervated gastrocnemius. For these same treatment groups, the citrate activation constant (Kₐ) was increased (P<0.01) from 3.6±0.3 to 13.2±1.1.

### Example 4

### Chronic Activation of AMPK in Denervated Rats

Rats were injected either with AICAR (1 mg in 0.9% NaCl/g body weight) or a 0.9% NaCl solution ˜0, ˜24, and ˜48 hours after denervation and were then sacrificed ˜72 hours after denervation without injection. Rats were anesthetized with sodium pentobarbital (4.8 mg/100 g. body weight), soleus and gastrocnemius muscles from both sham and denervated sides were extracted, and muscles were quick frozen in liquid nitrogen and stored for analysis.

Table 4 summarizes the findings relating to muscle atrophy. The denervated gastrocnemius and soleus muscles from both saline-treated (0.9% NaCl) and AICAR-treated (1 mg in 0.9% NaCl/g body weight) were significantly atrophied (P<0.05) as compared to the contralateral, innervated controls. Furthermore, the amount of atrophy was not significantly increased or decreased in the AICAR-treated muscles vs. the saline-treated muscles. The gastrocnemius muscles in both treatment groups lost ˜14% of their muscle mass after 3 days of denervation. The soleus muscles in both treatment groups lost ˜20% of their muscle mass after 3 days of denervation.

TABLE 4
Comparison of muscle weights in 3-day denervated and
contralateral control muscles from rats treated with
AICAR or .9% NaCl

Muscle Weight (mg)

| Treatment | Soleus | Gastrocnemius |
|---|---|---|
| Saline-treated Denervated | 64 ± 3.0* | 992 ± 31* |
| Saline-treated Control | 82 ± 4.4 | 1,158 ± 21 |
| AICAR-treated Denervated | 67 ± 3.0* | 920 ± 46* |
| AICAR-treated Control | 82 ± 4.4 | 1,055 ± 45 |

Values are means ± SE. *P < 0.05 control vs. denervated.

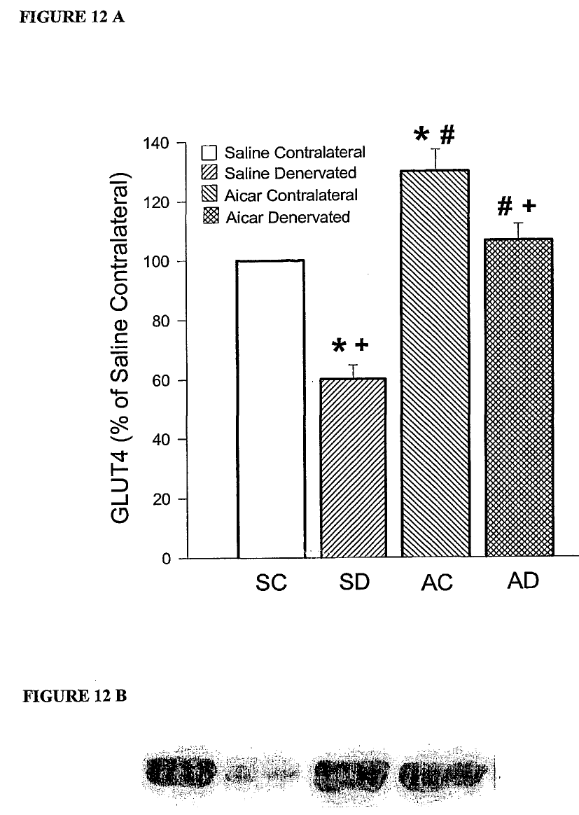

Total GLUT4 increases in muscles of denervated rats in response to 3 day AICAR injection. The bar graph and Western blot of FIGS. 12A and 12B displays GLUT4 content of denervated and contralateral gastrocnemius muscles from rats treated with 1 mg AICAR in 0.9% NaCl/g body weight (n=7 for each treatment group). Values are means±SE for 7 muscles per group. Values are expressed as percent intensity of saline-treated contralateral innervated controls. An asterisk indicates P<0.01 vs. saline contralateral, and a #P<0.01 vs. saline denervated. +P<0.01 vs. AICAR contralateral. GLUT4 content of denervated gastrocnemius muscles was found to be 60.1%±4.7 of GLUT4 content in the contralateral innervated gastrocnemius muscles. GLUT4 content in denervated gastrocnemius muscles treated with AICAR was significantly increased (106.6%±5.5) over GLUT4 levels in denervated gastrocnemius muscles treated with saline. Furthermore, the AICAR-treated contralateral innervated muscles contained significantly increased levels of GLUT4 (130.1%±7.2) when compared to saline-treated contralateral gastrocnemius muscles.

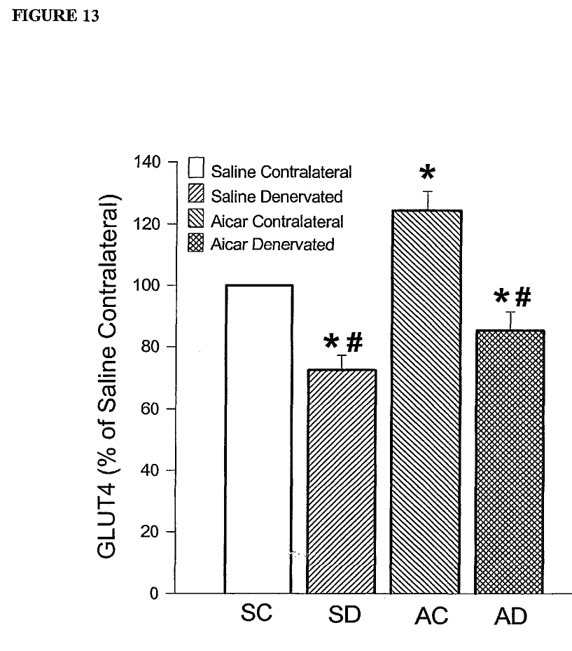

FIG. 13 exhibits GLUT4 content of denervated and contralateral soleus muscles from rats treated with 1 mg AICAR in 0.9% NaCl/g body weight or with just 0.9% NaCl (n=7 for each treatment group). Values are means±SE for 7 muscles per group. An asterisk indicates P<0.01 vs. saline contralateral, and a # indicates P<0.01 vs. AICAR contralateral. Although the loss of GLUT4 content of the denervated soleus (72.6%±4.7) was significant, GLUT 4 content neither decreases to the extent that GLUT4 decreased in the denervated gastrocnemius in response to denervation, nor does GLUT4 content significantly increase (P<0.05) in the denervated soleus in response to AICAR (85.4%±6.1) vs. saline-treated, denervated soleus muscles.

### Example 5

### Dose Response of AMPK with Activation by AICAR in Denervated Rats

Rats were denervated as described above between 8:00 and 10:00 and were immediately injection with either 0.9% NaCl solution (n=4), 0.1 mg AICAR in 0.9% NaCl/g body weight (n=4), 0.5 mg AICAR in 0.9% NaCl/g body weight (n=3), or 1 mg AICAR in 0.9% NaCl/g body weight (n=4). Rats of each treatment group were subsequently injected with the respective AICAR solutions at ˜24 and ˜48 hours after denervation. Rats were killed ˜72 hours after denervation without an AICAR injection and muscles were collected as described above.

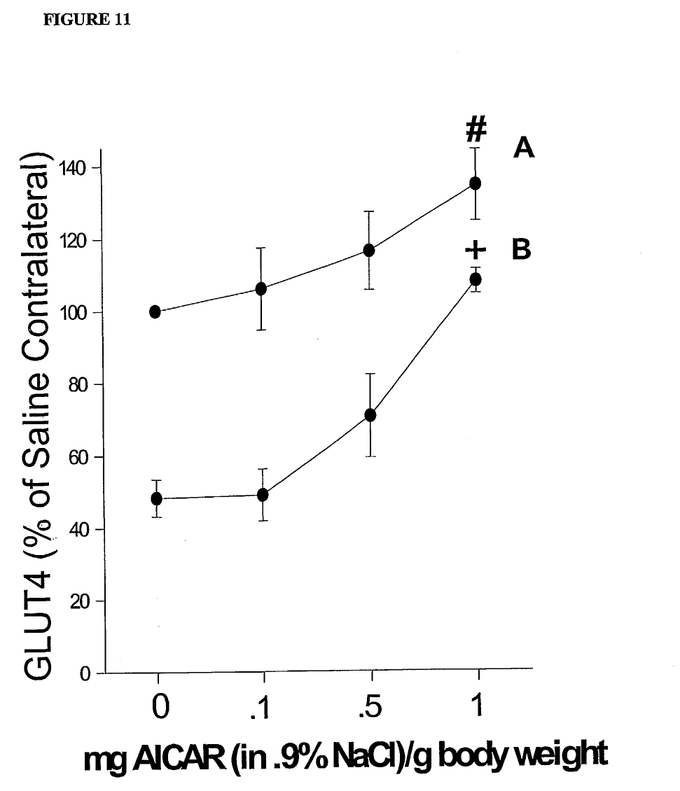

FIG. 11 shows the dose dependent response of GLUT4 in AICAR-treated denervated gastrocnemius muscles (curve B) and AICAR-treated contralateral innervated gastrocnemius muscles (curve A). Values are expressed as percent intensity of saline-treated contralateral innervated controls. Values are means±SE. +P<0.05 vs. saline-treated denervated muscle. A # indicates P<0.05 vs. saline-treated contralateral innervated control. For all GLUT4 data all saline-treated, contralateral, innervated muscles were normalized to 100%. Data from all other muscles were compared to the saline-treated, contralateral muscles and expressed as a percent of this control. The 1 mg AICAR in 0.9% NaCl/g body weight dose is shown to be the only dose that significantly raises GLUT4 content in denervated gastrocnemius muscles above the GLUT4 content in the saline-treated, denervated gastrocnemius muscles (P<0.05). The 1 mg AICAR in 0.9% NaCl/g body weight dose is also shown to be the only dose that significantly raises GLUT4 content in innervated gastrocnemius muscles above the GLUT4 content in saline-treated innervated gastrocnemius muscles (P<0.05).

### SUMMARY

In summary, chronic AMPK activation using AICAR injection in resting rats results in significant increases in ALA synthase, cytochrome c, citrate synthase, malate dehydrogenase, in white quadriceps, but not in red quadriceps. Hexokinase activity was significantly increased in all three muscle types. GLUT4 was increased in both red and white quadriceps, and a trend toward an increase was noted in soleus. The extent of AMPK activation appeared to be greatest in the white quadriceps, evidenced by AMPK activity measurements in the presence of maximally effective AMP concentrations and by extent of reduction in ACC activity (target for AMPK). These results suggest that the activation of AMPK that accompanies muscle contraction during the daily bouts of training may play a role in mediating some of the biochemical adaptations that are induced in skeletal muscle by endurance exercise. The data also suggest that not all muscular adaptations to training are mediated by activation of this kinase. In addition, the chronic activation of AMPK via subcutaneous injection of AICAR in denervated skeletal muscles leads to significant increases in total GLUT4 in denervated gastrocnemius muscles but not in denervated soleus muscles. These data are consistent wit the proposals that chemical activation of AMPK may be an effective approach for treating glucose transport impairments.

The invention may be embodied in other specific forms without departing from its essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope. All patents, publications, and commercial materials cited herein are hereby incorporated by reference.

1. A method for stimulating AMP-activated protein kinase in muscle of a mammal comprising:
administering a therapeutically effective amount of an AMP-activated protein kinase activator to the mammal.

2. The method of claim 1, wherein administering a therapeutically effective amount of an AMP-activated protein kinase activator results in an increased mitochondrial oxidative enzyme activity in the muscle of the mammal.

3. The method of claim 1, wherein the AMP-activated protein kinase activator is subcutaneously injected into the mammal.

4. The method of claim 1, wherein the AMP-activated protein kinase activator comprises an AMP analogue.

5. The method of claim 4, wherein the AMP analogue is selected from the group consisting of adenosine-5′-thiomonophosphate, adenosine 5′-phosphoramidate, formycin A 5′-monophosphate, and ZMP.

6. The method of claim 5, wherein the AMP analogue is modified previous to administration to facilitate uptake by cells.

7. The method of claim 5, wherein the AMP analogue is administered intra-cellularly.

8. The method of claim 4, wherein the AMP analogue comprises 5-aminoimidazole-4-carboxamide ribonucleoside.

9. The method of claim 4, wherein 5-aminoimidazole-4-carboxamide ribonucleoside is administered at a dose from about 0.5 to at least about 1.0 mg/g body weight.

10. The method of claim 1, wherein the AMP-activated protein kinase activator is administered acutely.

11. The method of claim 1, wherein the AMP-activated protein kinase activator is administered chronically.

12. The method of claim 1, wherein the AMP-activated protein kinase activator is administered intermittently.

13. The method of claim 1, wherein the AMP-activated protein kinase activator comprises 5-aminoimidazole-4-carboxamide ribonucleoside.

14. The method of claim 1, wherein the administering a therapeutically effective amount of an AMP-activated protein kinase activator results in a reduction in body fat content of the mammal.

15. A method for increasing the mitochondrial oxidative enzyme activity in muscle of a mammal comprising:
administering a therapeutically effective amount of an AMP-activated protein kinase activator to the mammal.

16. The method of claim 15, wherein the AMP-activated protein kinase activator is subcutaneously injected into the mammal.

17. The method of claim 15, wherein the AMP-activated protein kinase activator comprises an AMP analogue.

18. The method of claim 17, wherein the AMP analogue is selected from the group consisting of adenosine-5′-thiomonophosphate, adenosine 5′-phosphoramidate, formycin A 5′-monophosphate, and ZMP.

19. The method of claim 18, wherein the AMP analogue is modified previous to administration to facilitate uptake by cells.

20. The method of claim 18, wherein the AMP analogue is administered intra-cellularly.

21. The method of claim 17, wherein the AMP analogue comprises 5-aminoimidazole-4-carboxamide ribonucleoside.

22. The method of claim 17, wherein 5-aminoimidazole-4-carboxamide ribonucleoside is administered at a dose from about 0.5 to at least about 1.0 mg/g body weight.

23. The method of claim 15, wherein the AMP-activated protein kinase activator is administered acutely.

24. The method of claim 15, wherein the AMP-activated protein kinase activator is administered chronically.

25. The method of claim 15, wherein the AMP-activated protein kinase activator is administered intermittently.

26. The method of claim 15, wherein the AMP-activated protein kinase activator comprises 5-aminoimidazole-4-carboxamide ribonucleoside.

27. The method of claim 15, wherein the administering a therapeutically effective amount of an AMP-activated protein kinase activator results in a reduction in body fat content of the mammal.

28. A method of treating obesity in a mammal comprising administering a therapeutically effective amount of an AMP-activated protein kinase activator to the mammal.

29. The method of claim 28, wherein the administering a therapeutically effective amount of an AMP-activated protein kinase activator results in an increased mitochondrial oxidative enzyme activity in the muscle of the mammal.

30. The method of claim 28, wherein the AMP-activated protein kinase activator is subcutaneously injected into the mammal.

31. The method of claim 28, wherein the AMP-activated protein kinase activator comprises an AMP analogue.

32. The method of claim 31, wherein the AMP analogue is selected from the group consisting of adenosine-5′-thiomonophosphate, adenosine 5′-phosphoramidate, formycin A 5′-monophosphate, and ZMP.

33. The method of claim 32, wherein the AMP analogue is modified previous to administration to facilitate uptake by cells.

34. The method of claim 32, wherein the AMP analogue is administered intra-cellularly.

35. The method of claim 31, wherein the AMP analogue comprises 5-aminoimidazole-4-carboxamide ribonucleoside.

36. The method of claim 35, wherein 5-aminoimidazole-4-carboxamide ribonucleoside is administered at a dose from about 0.5 to at least about 1.0 mg/g body weight.

37. The method of claim 28, wherein the AMP-activated protein kinase activator is administered acutely.

38. The method of claim 28, wherein the AMP-activated protein kinase activator is administered chronically.

39. The method of claim 28, wherein the AMP-activated protein kinase activator is administered intermittently.

40. The method of claim 28, wherein the AMP-activated protein kinase activator comprises 5-aminoimidazole-4-carboxamide ribonucleoside.

41. The method of claim 28, wherein the administering a therapeutically effective amount of an AMP-activated protein kinase activator results in a reduction in body fat.

42. A method of treating insulin resistance in a mammal suffering from obesity comprising administering a therapeutically effective amount of an AMP-activated protein kinase activator.

43. The method of claim 42, wherein the AMP-activated protein kinase activator comprises an AMP analogue.

44. The method of claim 43, wherein the AMP analogue comprises 5-aminoimidazole-4-carboxamide ribonucleoside.

45. The method of claim 42, wherein the AMP-activated protein kinase activator comprises 5-aminoimidazole-4-carboxamide ribonucleoside.

46. A method of treating insulin resistance in a mammal suffering from type 2 diabetes comprising administering a therapeutically effective amount of an AMP-activated protein kinase activator.

47. The method of claim 46, wherein the AMP-activated protein kinase activator comprises an AMP analogue.

48. The method of claim 43, wherein the AMP analogue comprises 5-aminoimidazole-4-carboxamide ribonucleoside.

49. The method of claim 42, wherein the AMP-activated protein kinase activator comprises 5-aminoimidazole-4-carboxamide ribonucleoside.

50. A method of treating insulin resistance in a mammal suffering from muscle paralysis comprising administering a therapeutic amount of an AMP-activated protein kinase activator.

51. The method of claim 50, wherein the AMP-activated protein kinase activator comprises an AMP analogue.

52. The method of claim 51, wherein the AMP analogue comprises 5-aminoimidazole-4-carboxamide ribonucleoside.

53. The method of claim 50, wherein the AMP-activated protein kinase activator comprises 5-aminoimidazole-4-carboxamide ribonucleoside.

54. An ergogenic aid comprising a therapeutically effective amount of an AMP-activated protein kinase activator.

55. The ergogenic aid of claim 54, wherein the AMP-activated protein kinase activator comprises an AMP analogue.

56. The ergogenic aid of claim 55, wherein the AMP analogue comprises 5-aminoimidazole-4-carboxamide ribonucleoside.

57. The ergogenic aid of claim 54, wherein the AMP-activated protein kinase activator comprises 5-aminoimidazole-4-carboxamide ribonucleoside.
